(19) United States
(12) Patent Application Publication
(10) Pub. No.: US 2025/0098972 A1
(43) Pub. Date: Mar. 27, 2025

(54) METHOD AND APPARATUS FOR PROBE-ADAPTIVE QUANTITATIVE ULTRASOUND IMAGING

(71) Applicant: KOREA ADVANCED INSTITUTE OF SCIENCE AND TECHNOLOGY, Daejeon (KR)

(72) Inventors: Hyeon-Min BAE, Daejeon (KR); Seokhwan OH, Daejeon (KR); Myeong Gee KIM, Daejeon (KR); Youngmin KIM, Daejeon (KR); Guil JUNG, Daejeon (KR)

(21) Appl. No.: 18/964,080

(22) Filed: Nov. 29, 2024

Related U.S. Application Data
(63) Continuation of application No. PCT/KR2023/007267, filed on May 26, 2023.

(30) Foreign Application Priority Data
Jun. 3, 2022 (KR) 10-2022-0068239

(51) Int. Cl. A61B 5/05

(52) U.S. Cl. CPC A61B 5/05

(57) ABSTRACT

Disclosed is an operating method of an apparatus operated by at least one processor, which includes: receiving RF data obtained from tissue through an arbitrary ultrasound probe; extracting a generalized quantitative feature to a probe domain from the RF data; and reconstructing the generalized quantitative feature to generating a quantitative ultrasound image.

### CROSS-REFERENCE TO RELATED APPLICATION

This application claims priority to and the benefit of Korean Patent Application No. 10-2022-0068239 filed in the Korean Intellectual Property Office on Jun. 3, 2022, and International application No. PCT/KR2023/007267 filed on May 26, 2023, the entire contents of which are incorporated herein by reference.

### BACKGROUND

### (a) Field

The present disclosure relates to artificial intelligence-based quantitative ultrasound imaging technology.

### (b) Description of the Related Art

Cancer is challenging to detect in its early stage, requiring regular diagnosis and continuous monitoring of lesion size and characteristics. Representative imaging modalities for this includes X-ray, magnetic resonance imaging (MRI), computed tomography (CT), and ultrasound. While the X-ray, the MRI, and the CT have drawbacks of radiation exposure, long measurement times, and high costs, the ultrasound imaging is safe, more affordable, and provides real-time imaging. This enables users to monitor lesions in real time and obtain the desired image.

A brightness mode (B-mode) ultrasound imaging is a method of determining the location and size of an object by measuring the time and intensity of ultrasound waves reflected from the surface of the object. This method allows real-time lesion detection, enabling users to monitor lesions in real time and efficiently obtain desired images. It is also safe, relatively inexpensive, and widely accessible. However, the method has a limitation in that the method provides only qualitative information that is user-dependent and does not provide tissue characteristics.

To overcome the clinical limitation, a need for quantitative ultrasound imaging technology is increasing, and methods for extracting quantitative information using deep learning technology are under research. However, the performance of a neural network in real-world applications depends on the similarity between the training conditions and the real-world conditions. There is a limitation that the reliability of the quantitative information extraction method using the deep learning technology is not guaranteed because ultrasound probes of various shapes, differing from the training condition, are used in the real-world application environment such as hospitals.

### SUMMARY

The present disclosure attempts to provide a method and an apparatus for probe-adaptive quantitative ultrasound imaging that generate a quantitative ultrasound image based on a neural network, regardless of a probe used to obtain RF data.

The present disclosure attempts to provide a neural network that generates augmented data related to virtual probe conditions and generalizes a probe domain through meta-learning using the augmented data to extract a quantitative feature.

An exemplary embodiment of the present disclosure provides an operating method of an apparatus operated by at least one processor. The operating method comprises: receiving RF data obtained from tissue through an arbitrary ultrasound probe; and generating a quantitative ultrasound image from the RF data using a neural network trained to perform a probe domain generalization.

The generating the quantitative ultrasound image may comprises extracting a generalized quantitative feature from the RF data using a calibration function that meta-learns probe domain generalization; and reconstructing the generalized quantitative feature to generate the quantitative ultrasound image.

The calibration function may generate a deformation field spatially transforming a probe condition of the arbitrary ultrasound probe to a generalized probe condition.

The generating the quantitative ultrasound image may comprise applying the deformation field generated by the calibration function to a feature of the RF data to generate a deformed feature to the generalized probe condition.

The operation method may further comprise generating a B-mode image from the RF data. The generating the quantitative ultrasound image may comprise generalizing the probe condition inferred from a relationship between the RF data and the B-mode image, using the calibration function; and extracting the generalized quantitative feature from the RF data.

The quantitative ultrasound image may include quantitative information for at least one parameter among speed of sound (SoS), attenuation coefficient (AC), effective scatterer Concentration (ESC), and effective scatterer diameter (ESD).

The neural network may be an artificial intelligence model trained to generalize the probe domain of input RF data using training data augmented with virtual probe conditions.

Another exemplary embodiment of the present disclosure provides an operating method of an apparatus operated by at least one processor. The operating method comprises: augmenting source training data with virtual data related to virtual probe conditions; and training a neural network using data-augmented training data, the neural network trained to generate a quantitative ultrasound image from an input RF data by a probe domain generalization.

The augmenting the source training data may comprise generating new virtual data by changing at least one of the number of sensors of a probe, a pitch between sensors, a sensor width and sensor frequency in the source training data.

The training the neural network may comprise training a calibration function in the neural network, through meta-learning using the data-augmented training data, the calibration function configured to generate a deformation field corresponding to a probe condition of the input RF data. The calibration function may be trained to generates the deformation field spatially transforming a probe condition of an arbitrary ultrasound probe to a generalized probe condition.

The calibration function may perform the meta-learning that generates the deformation fields spatially transforming the probe condition of the input RF data to a generalized probe condition, based on a relationship between the input RF data and a B-mode image generated from the input RF data. The training the neural network may comprise training the neutral network to minimize a loss of an interfered quantitative ultrasound image while performing the meta-learning of the calibration function. The neural network may include: an encoder extracting a quantitative feature generalized to a probe domain from the input RF data using an adaptation module that generalizes the probe condition of the input RF data; and a decoder reconstructing the generalized quantitative feature to generate the quantitative ultrasound image.

Yet another exemplary embodiment of the present disclosure provides an imaging apparatus comprising: a memory; and a processor executing instructions loaded to the memory. The processor is configured to: receive RF data obtained from tissue through an arbitrary ultrasound probe; and generate a quantitative ultrasound image by performing a probe domain generalization for the RF data using a trained neural network.

The processor may be configured to: extract a generalized quantitative feature from the RF data using a calibration function that meta-learns probe domain generalization; and reconstruct the generalized quantitative feature to generate the quantitative ultrasound image.

The calibration function may generate a deformation field spatially transforming a probe condition of the arbitrary ultrasound probe to a generalized probe condition.

The processor may be configured to apply the deformation field generated by the calibration function to a feature of the RF data to generate a deformed feature to the generalized probe condition. The processor may be configured to: generate a B-mode image from the RF data; and generalize a probe condition inferred from a relationship between the RF data and the B-mode image using the calibration function, and then extract the generalized quantitative feature from the RF data.

The quantitative ultrasound image may include quantitative information for at least one parameter among speed of sound (SoS), attenuation coefficient (AC), effective scatterer concentration (ESC), and effective scatterer diameter (ESD).

The neural network may include: an encoder extracting a quantitative feature generalized to a probe domain from the input RF data using an adaptation module that generalizes the probe condition of the input RF data; and a decoder reconstructing the generalized quantitative feature to generate the quantitative ultrasound image.

According to exemplary embodiments, consistent quantitative ultrasound images can be generated regardless of probe conditions, even when various types of probes are used in real-world application environments. Therefore, according to exemplary embodiments, in the field of ultrasound-based diagnostic technology, the clinical usability of quantitative information such as attenuation coefficients extracted based on artificial intelligence can be increased.

According to exemplary embodiments, a neural network that generates quantitative ultrasound images can be domain-generalized so that the neural network can be used for probe conditions that is not seen during the training process.

According to exemplary embodiments, the quantitative ultrasound images can be generated using various types of ultrasound probes and imaging devices for B-mode imaging as they are.

### BRIEF DESCRIPTION OF THE DRAWINGS

FIG. 1 is a diagram conceptually describing a quantitative ultrasound imaging apparatus according to an exemplary embodiment.

FIG. 2 is a conceptual view of a neural network according to an exemplary embodiment.

FIG. 3 illustrates a network architecture of an encoder according to an exemplary embodiment.

FIG. 4 illustrates an example of an adaptation module according to an exemplary embodiment.

FIG. 5 illustrates a network architecture of a decoder according to an exemplary embodiment.

FIG. 6 is a diagram describing data augmentation related to virtual probe conditions according to an exemplary embodiment.

FIG. 7 is a diagram describing a calibration function training method according to an exemplary embodiment.

FIG. 8 is a diagram describing a neural network training method according to an exemplary embodiment.

FIG. 9 is a flowchart of a neural network training method according to an exemplary embodiment.

FIG. 10 is a flowchart of a probe-adaptive quantitative ultrasound imaging method according to an exemplary embodiment.

FIG. 11A and FIG. 11B illustrate quantitative imaging results

FIG. 12A and FIG. 12B are graphs comparing the consistency of quantitative information.

FIG. 13 is a configuration diagram of a computing device according to an exemplary embodiment.

### DETAILED DESCRIPTION OF THE EMBODIMENTS

In the following detailed description, only certain exemplary embodiments of the present disclosure have been shown and described, simply by way of illustration. However, the present disclosure can be variously implemented and is not limited to the following exemplary embodiments. In addition, in the drawings, the drawings and description are to be regarded as illustrative in nature and not restrictive. Like reference numerals designate like elements throughout the specification.

Through the specification, unless explicitly described to the contrary, the word “comprise”, and variations such as “comprises” or “comprising”, will be understood to imply the inclusion of stated elements but not the exclusion of any other elements. In addition, the terms “-er”, “-or”, and “module” described in the specification mean units for processing at least one function and operation, and can be implemented by hardware components or software components, and combinations thereof.

The device of the present disclosure is a computing device configured and connected so that at least one processor may perform the operation of the present disclosure by executing instructions. The computer program may include instructions described for the processor to execute the operation of the present disclosure, and may be stored in a non-transitory computer readable storage medium. The computer program may be downloaded through a network or sold in a product form.

A neural network of the present disclosure is an artificial intelligence (AI) model that learns at least one task, and may be implemented as software/computer program executed in a computing device. The neural network may be downloaded through a communication network or sold in the product form. Alternatively, the neural network can interlock with various devices via the communications network.

In the present disclosure, domain generalization means processing data so as not to be affected by characteristics of a domain from which the data is collected, thereby making it impossible to distinguish which domain the data is collected from, and in the present disclosure, the domain may be a probe domain from which data is obtained.

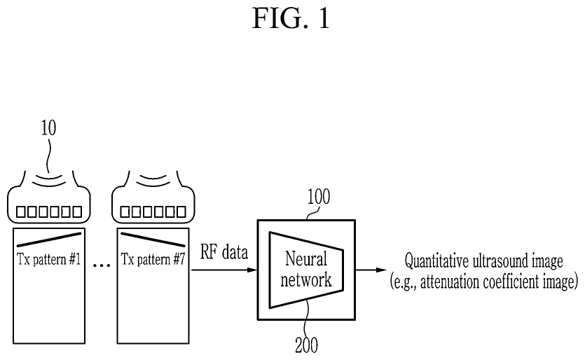

FIG. 1 is a diagram conceptually describing a quantitative ultrasound imaging apparatus according to an exemplary embodiment.

Referring to FIG. 1, the quantitative ultrasound imaging apparatus (referred to simply as an “imaging apparatus”) 100 is a computing device operated by at least one processor, and is equipped with a computer program that performs the operations described in the present disclosure, and the computer program is executed by the processor. A neural network 200 implemented on the imaging apparatus 100 as an artificial intelligence model capable of learning at least one task, may be realized as software/program executed in the computing device.

The imaging apparatus 100 receives radio frequency (RF) data obtained from tissue through an ultrasound probe 10 and uses the neural network 200 to extract quantitative information of the tissue. The quantitative information of the tissue may be represented in a quantitative ultrasound image. The quantitative ultrasound image may be referred to simply as a “quantitative image”. The quantitative image may include quantitative information for at least one parameter, such as attenuation coefficient (AC), speed of sound (SoS), effective scatterer concentration (ESC) which indicates the density distribution within the tissue, and effective scatterer diameter (ESD) which indicates the size of cells in the tissue as quantitative parameters of the tissue. In the description, an attenuation coefficient image may be used as an example of the quantitative image.

The neural network 200 may be implemented on the imaging apparatus 100 after being trained by a separate training apparatus. For convenience of description, it may be described that the imaging apparatus 100 generates training data and trains the neural network 200 based on the training data.

An implementation form of the imaging apparatus 100 may vary. For example, the imaging apparatus 100 may be mounted on an image capturing apparatus.

Alternatively, the imaging apparatus 100 may be constructed as a server apparatus that interfaces with at least one image capturing apparatus. The imaging apparatus 100 may be a local server connected to a communication network within a specific medical institution or a cloud server interfacing with devices across multiple medical institutions with access permissions.

The ultrasound probe 10 may sequentially emit ultrasound signals with different beam patterns (Tx patterns #1 to #k) into the tissue and receive RF data reflected back from the tissue. RF data obtained using a plurality of beam patterns may also be referred to as pulse-echo data or beamformed ultrasound data. For example, the RF data may be acquired from plane waves with seven different incident angles (θ1 to θ7). The incident angles may be set to, for example, −15°, −10°, −5°, 0°, 5°, 10°, and 15°. The ultrasound probe 10 is composed of N sensor elements arranged at regular intervals. The sensor elements can be implemented as piezoelectric elements.

Meanwhile, ultrasound probes used to obtain RF data may be manufactured by various manufacturers and have diverse probe conditions. Here, the probe conditions may vary depending on a sensor geometry, such as the number of sensors, the pitch between sensors, the sensor width and sensor frequency. Since it is impractical to use RF data obtained from all types of ultrasound probes in neural network training, ultrasound probes used in clinical practice often differ from those used in the neural network training. Consequently, in real-world clinical environments such as hospitals, an expected performance of the neural network may not be provided due to the mismatches of the ultrasound probes used to obtain the RF data.

To address performance degradation under unseen probe conditions, the neural network 200 has a network architecture that generalizes the probe domain from which RF data is obtained to extract quantitative features and reconstructs the generalized quantitative features to generate a quantitative image. The neural network 200 may find a deformation field for spatially transforming a probe condition of input RF data into a generalized probe condition, calibrate the input RF data using the deformation field, and then extract the quantitative features.

The neural network 200 may generalize probe geometries that varies depending on various probe conditions, such as the number of sensors, the pitch between sensors, the sensor width and sensor frequency, through learning using a dataset that includes a variety of probe conditions. In this case, the neural network 200 may perform learning for probe domain generalization based on data augmented with various new virtual probe conditions, the virtual probe conditions generated from a source dataset. By the dataset augmented with various new probe conditions, probe generalization performance may be enhanced. Here, the neural network 200 may learn a calibration function that generates a deformation field for a probe condition through meta-learning using augmented data. The probe condition may be inferred from a relationship between the RF data and the B-mode image generated from the RF data. Accordingly, the neural network 200 may have a network architecture that extracts the deformation field corresponding to the probe condition by using both the B-mode image and the RF data.

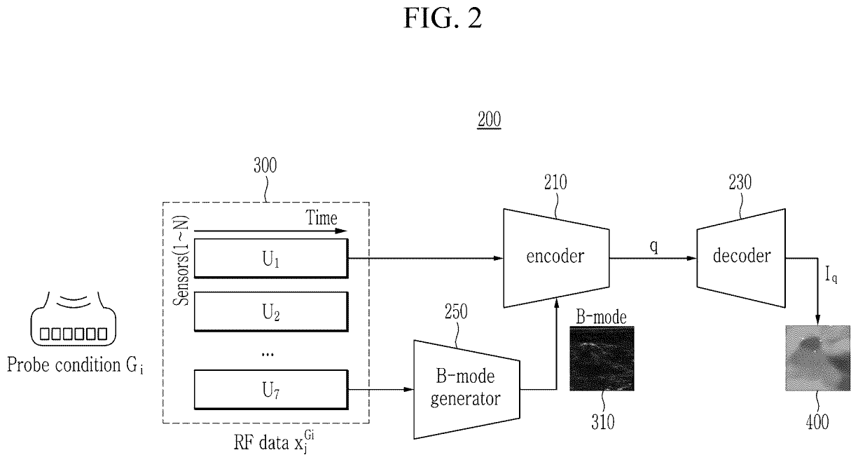

FIG. 2 is a conceptual view of a neural network according to an exemplary embodiment.

Referring to FIG. 2, the neural network 200 receives RF data 300 obtained by an ultrasound probe Gᵢ, and reconstructs quantitative information of RF data xⱼ^{Gi}, and outputs a quantitative ultrasound image 400. The RF data 300 is data obtained by sequentially emitting ultrasound signals with different beam patterns to the tissue by the ultrasound probe. The RF data 300 may be, for example, RF data (U₁ to U₇) obtained from seven distinct beam patterns (θ₁ to θ₇) and includes information received from sensors of the ultrasound probe at time indices. In this case, despite being obtained from the same tissue, the RF data may differ depending on the probe condition Gᵢ, so the neural network 200 performs probe domain generalization to reconstruct consistent quantitative information, regardless of the probe.

The neural network 200 may include an encoder 210 that extracts a quantitative feature q from the RF data 300 obtained under the probe condition Gᵢ, and a decoder 230 that reconstruct a quantitative feature q to generate a quantitative image I_{q} 400. In this case, the neural network 200 may further include a B-mode generator 250 that generates a B-mode image 310 from the RF data 300. The B-mode generator 250 may generate the B-mode image 310 by applying a delay and sum (DAS) and time gain compensation (TGC) to the RF data 300.

The encoder 210, a network trained to extract the quantitative feature q from the RF data 300 using convolution, extracts a generalized quantitative feature q by transforming RF data 300 obtained under an arbitrary probe condition into a generalized probe condition. To achieve this, the encoder 210 may find a deformation field corresponding to the input probe condition, calibrate the input data using the deformation field, and then extract the generalized quantitative feature, regardless of the probe. The probe condition may be inferred from the relationship between the RF data and the B-mode image generated from the RF data. Accordingly, the encoder 210 may receive the B-mode image 310 output from the B-mode generator 250, and infer the deformation field corresponding to the probe condition by using the relationship between the RF data and the B-mode image.

The decoder 230 transforms the quantitative feature q output from the encoder 210 into a high-resolution quantitative image 400. The decoder 230 may employ various network architectures and generate the high-resolution quantitative image, for example, by using High-Resolution Network (HRNet) based parallel multi-resolution subnetworks.

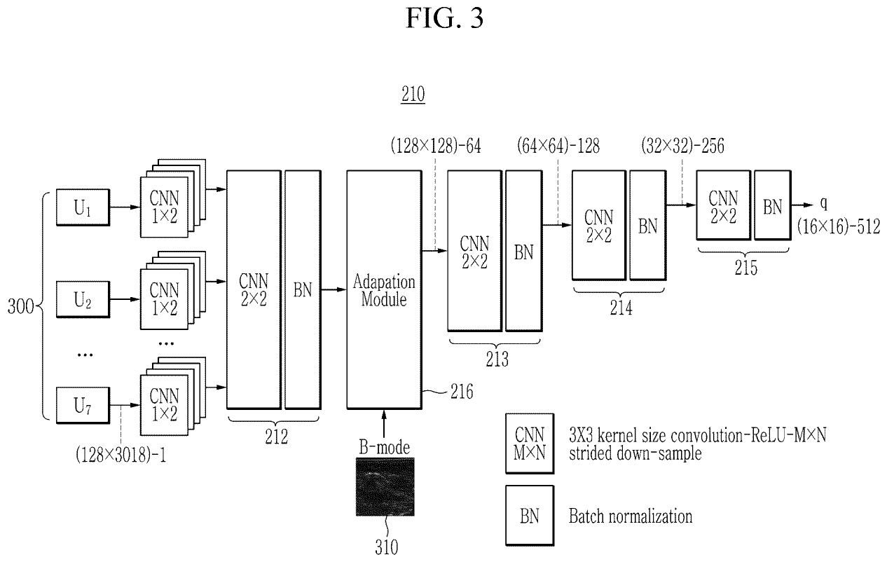

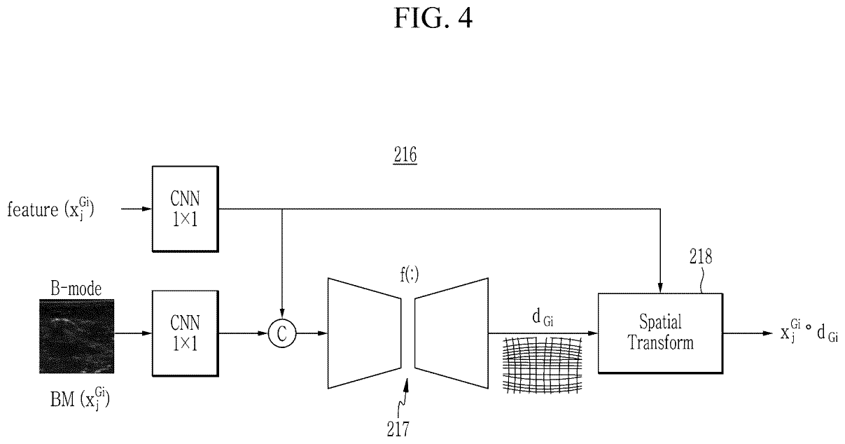

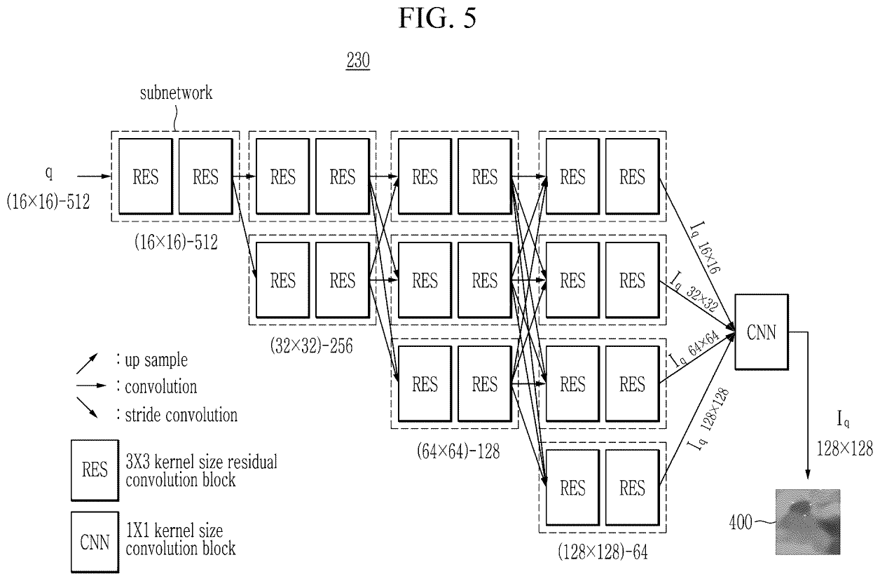

FIG. 3 illustrates a network architecture of an encoder according to an exemplary embodiment, FIG. 4 illustrates an example of an adaptation module according to an exemplary embodiment, and FIG. 5 illustrates a network architecture of a decoder according to an exemplary embodiment.

Referring to FIG. 3, the encoder 210 may be configured to various network architectures that receive RF data 300 related to the arbitrary probe condition among various probe conditions and extracts the generalized quantitative feature q.

For example, the encoder 210 may include a convolution-based individual encoding layer 211 that receives RF data U₁, U₂, . . . , U₇, and individually extracts features, and a plurality of convolution-based encoder layers 212, 213, 214, and 215 that connect the features extracted by the individual encoding layer 211, and encode the features.

The individual encoding layer 211 may be configured to perform convolution with 3×3 kernel size, apply an activation function (ReLU), and perform downsampling with 1×2 stride.

The plurality of encoding layers 212, 213, 214, and 215 are connected in series, and encodes input features to output a finally compressed quantitative feature q. The quantitative feature q may be represented with a spatial resolution R^{16×16×512}.

The encoder 210 includes an adaptation module 216 for transforming the RF data obtained under various probe conditions into the RF data obtained under the generalized probe condition. The adaptation module 216 may perform probe domain generalization for input data by calibrating the RF data obtained under the probe condition Gᵢ to the RF data obtained under the generalized probe condition. The adaptation module 216 may output the generalized feature using a relationship between an input feature output from a previous encoding layer and the B-mode image.

The adaptation module 216 may be placed after at least one of the encoding layers 212, 213, 214, and 215. The adaptation module 216 may be referred to as a deformable sensor adaptation (DSA) module.

Referring to FIG. 4, the adaptation module 216 may receive a B-mode image BM(xⱼ^{Gi}) together with the feature feature(xⱼ^{Gi}) of RF data xⱼ^{Gi} encoded in the previous encoding layer. The adaptation module 216 may include a deformation module 217 that generates a deformation field d_{Gi} from a relationship between the feature of the RF data and the B-mode image, and a spatial transformation module 218 that spatially transforms the input feature using the deformation field d_{Gi}. The deformation field d_{Gi} includes warping information for spatially transforming the probe condition Gᵢ into the generalized probe condition.

The deformation module 217 includes a calibration function f(:) that generates a deformation field d_{Gi} for generalizing the probe condition Gᵢ. The calibration function f(:) may generate a deformation field d_{Gi} according to a structural difference between the probe condition Gᵢ and the generalized probe condition through meta-learning using data augmented with virtual probe conditions. The calibration function f(:) may perform gradient based meta-learning. The deformation field d_{Gi} may be defined as shown in Equation 1 below. In Equation 1, BM(xc) may contribute to better recognize individual probe conditions Gᵢ.

$${d}_{G i} = Identity + f ( {x}_{j}^{G i} , BM ( {x}_{j}^{G i} ) ) \qquad ( Equation l )$$

The spatial transformation module 218 warps the input feature from the previous encoding layer through the deformation field d_{Gi} to output a deformation feature xⱼ^{Gi}·d_{Gi}. The deformation feature is a generalized feature regardless of the probe condition.

Referring to FIG. 5, the decoder 230 receives the quantitative feature q output from the encoder 210, and gradually synthesizes the quantitative feature q to output the high-resolution quantitative image 400. The decoder 230 may generate the high-resolution quantitative image using high-resolution network (HRNet) based parallel multi-resolution subnetworks. The subnetwork may include, for example, at least one residual convolution block.

A corresponding resolution image, for example, I_{q.16×16}, I_{q.32×32}, I_{q.64×64}, and I_{q.128×128} are generated in respective parallel networks of the decoder 230, which are subjected to convolution to finally output the quantitative image I_{q}.

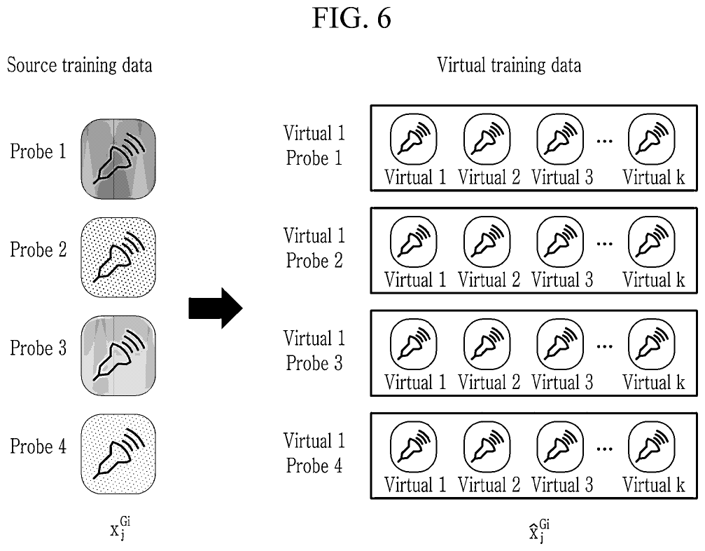

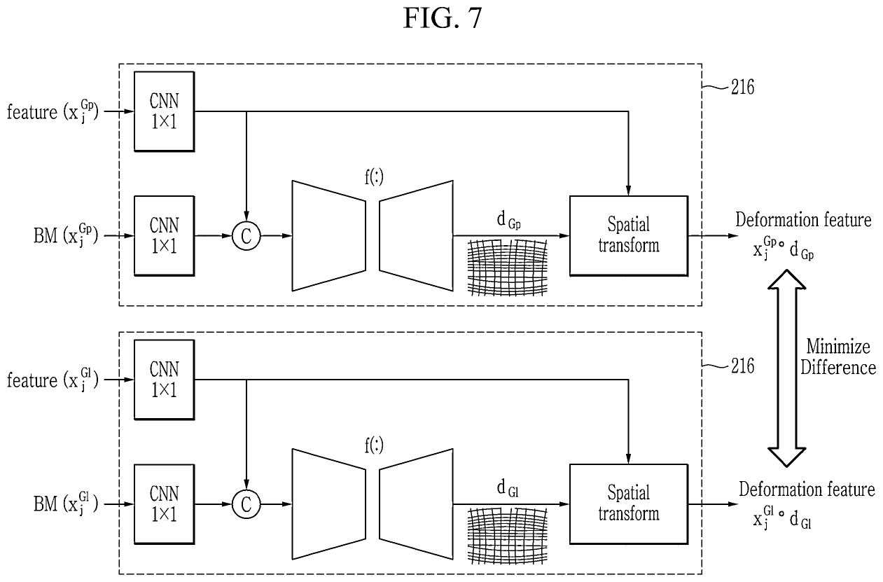

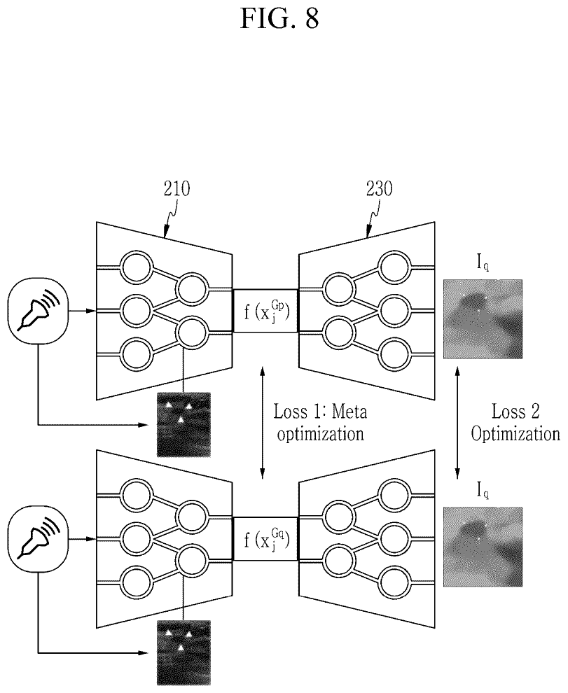

FIG. 6 is a diagram describing data augmentation related to virtual probe conditions according to an exemplary embodiment, FIG. 7 is a diagram describing a calibration function training method according to an exemplary embodiment, and FIG. 8 is a diagram describing a neural network training method according to an exemplary embodiment.

Referring to FIG. 6, the dataset may be acquired in various way. For example, source training data may consist of RF data obtained through a simulation phantom, and collected using an ultrasound simulation tool (e.g., k-wave toolbox of Matlab). For example, in the simulation phantom, organs and lesions yᵢ may be represented by placing 0 to 10 ellipses with a radius of 2 to 30 mm at random positions on a 50×50 mm background.

For training across various probe conditions, virtual training data generated by augmenting the source training data is used. A variety of data augmentation algorithm may be used for this purpose. For example, the data augmentation algorithm may generate RF data \hat{x}ⱼ^{Gi} which is virtual training data measured under a virtual probe condition based on source training data D. By using the augmented data, the neural network 200 may be trained to better adapt to a wide range of probe conditions and unseen sensor geometries.

The data augmentation algorithm adjusts the probe geometry (e.g., the number of virtual sensors and the sensor width) for each virtual probe by using hyperparameters, such as a sub-sample αₛₛ and a sub-width α_{sw}, the hyperparameters used to generate new various virtual probe datasets.

The data augmentation algorithm may randomly generate the sub-sample αₛₛ and sub-width α_{sw} parameters in a uniform distribution, and generate virtual training data \hat{x}ⱼ^{Gi} augmented from source training data xⱼ^{Gi} by using randomly generated probe conditions as in Table 1.

TABLE 1
Algorithm 1 Virtual sensor geometry augmentation

Input: Source training sample (xⱼ^{G}^{ᵢ}, yⱼ) ~ [symbol] ⊂( [symbol] , [symbol] , ... [symbol] , yⱼ) ~ [symbol]
Output: Virtual training sample (\hat{x}ⱼ^{G}^{ᵢ}, yⱼ) ~ [symbol]
Require: αₛₛ: Sub-sample hyper-parameter, α_{sw}: Sub-width hyper-parameter
1: Choose αₛₛ in uniform distribution (0.5, 1)
2: for t in 1:T do
$3 : {\overline{x}}_{j}^{{𝔾}_{i}} ( : \text{,} t ) ← {x}_{j}^{{𝔾}_{i}} ( round ( \frac{1 : {\overline{n}}_{element}}{{α}_{ss}} ) , t )$
4: end for
5: Choose α_{sw} in uniform distribution (0.7, 1)
6: for t in 1:T do
$7 : {\hat{x}}_{j}^{{𝔾}_{i}} ( : \text{,} t ) ← {\overline{x}}_{j}^{{𝔾}_{i}} ( \frac{{\overline{N}}_{element} ( 1 - {α}_{sw} )}{2} : \frac{{\tilde{N}}_{element} ( 1 - {α}_{sw} )}{2} , t ) )$
8: end for

Referring to FIG. 7, the calibration function f(:) of the neural network 200 may perform meta-learning to generate a deformation field d_{Gi} corresponding to the probe condition Gᵢ by using data augmented with the virtual probe conditions. In this case, the calibration function f(:) may be trained to generate deformation fields for RF data under different probe conditions Gₚ and Gₗ, and to minimize the Euclidian distance between two deformation features that are corrected using their respective deformation fields. This process may be referred to as Meta-Learned Spatial Deformation (MLSD).

Through this, even if RF data input to the neural network 200 is obtained under various probe conditions, a quantitative feature may be extracted by spatially transforming the RF data into a generalized probe condition. Accordingly, the neural network 200 may generate a consistent quantitative ultrasound image, regardless of probe conditions, even when various types of probes are used in real-world clinical environments.

A data-based approach may overfit to training conditions, resulting in poor performance on unseen application conditions. To address this, the meta-learning may be used to allow domain generalization to the unseen conditions in the neural network. That is, through the meta-learning, the calibration function is optimized to improve the generalizability of the adaptation module 216.

Referring to FIG. 8 and Table 2, the neural network 200, consisting of the encoder 210 and the decoder 230, may optimize the calibration function f(:) within the encoder 210 through the meta-learning.

Specifically, a training apparatus (not illustrated) allocates data D to meta-training data D̅ and meta-test data \hat{D}, and then trains the calibration function with the meta-training data to allow the calibration function to be generalized to the meta test data. At each iteration, the calibration function f is updated to f′ which minimizes the loss 1. The training apparatus iterates training to allow the adaptation module 216 to correct an input feature by appropriately spatially transforming the unseen probe condition without biasing for data.

While performing the meta-learning for the calibration function f, the neural network 200 may be trained to minimize a loss 2 for an output θ.

TABLE 2
Algorithm 2 Meta-learned spatial deformation

Input: ( [symbol] , [symbol] , ... [symbol] , yⱼ) ~ [symbol]
Initialize: DSG-net model parameter θ, DSA module paramter: f
Require: Hyper-parameter: β, λ, γ = (1e⁻⁴, 1e⁻⁴, 1)
1: for it in iterations do
2: Split [symbol] and [symbol] ← [symbol]
3: Meta-train: Loss L_{DSA} (f, [symbol] )
$4 : Update DSA parameter {f}^{′} = f - {β▽}_{f} = f - β \frac{\partial ( {L}_{DSA} ( f , \overline{𝔻} ) )}{\partial f}$
5: Meta-test: Meta DSA Loss L_{DSA}(f ′, [symbol] , [symbol] )
$6 : Meta ‐ DSA Optimization : f = f - λ \frac{\partial ( ( {L}_{DSA} ( f , \overline{𝔻} ) ) + γ {L}_{DSA} ( f - {β▽}_{f} , \overline{𝔻} , \hat{𝔻} ) )}{\partial f}$
$7 : Model Optimization : θ = θ - λ \frac{\partial {L}_{model} ( f , 𝔻 )}{\partial θ}$
8: end for

An objective function f* for the calibration function f may be defined as in Equation 2. In Equation 2, L_{DSA}(f,x^{p},x^{l}) represents the Euclidian distance between features in which the meta-training data x^{p} and x^{l} are deformed through the calibration function f. L_{DSA}(f′,x^{p},x^{r}) represents the Euclidian distance between features in which the meta-training data x^{p} and the meta-test data x^{r} are deformed through the updated calibration function f′. That is, f* represents the objective function that minimizes the Euclidian distance between the deformation features for the data.

$${f}^{*} = {arg min}_{f} {𝔼}_{( {x}^{p} , {x}^{l} , y ) ⊂ \overline{𝔻} , ( {x}^{r} , y ) ⊂ \breve{𝔻}} [ {L}_{DSA} ( f , {x}^{p} , {x}^{l} ) + {L}_{DSA} ( {f}^{′} , {x}^{p} , {x}^{r} ) ] , \\ {L}_{DSA} ( f , x , y ) = {‖ f ( x ) - f ( y ) ‖}^{2} , \qquad ( Equation 2 )$$

An objective function θ* of the neural network 200 may be defined as in Equation 3. While an objective function OR of each parallel network of the decoder 230 is normalized to gradually generate a corresponding resolution image y_{R}, θ* is an objective function that minimizes a loss between a ground truth y and an output θ(x). The ground truth y is a ground truth quantitative image, and the output θ(x) is a quantitative image reconfigured from input RF data x.

$${{θ}^{*} = {arg min}_{θ} {𝔼}_{( x , y ) ⊂ 𝔻} [ ‖ y - θ ( x ) ) ‖}^{2} ] + {{\sum }_{R} [ ‖ {y}_{R} - {θ}_{R} ( x ) ) ‖}^{2} ] + ϵ {\sum }_{i = 1} {w}_{i}^{2} \qquad ( Equation 3 )$$

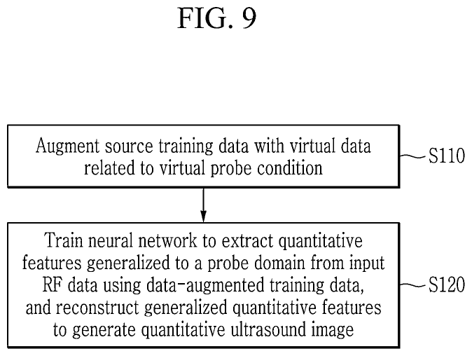

FIG. 9 is a flowchart of a neural network training method according to an exemplary embodiment.

Referring to FIG. 9, the imaging apparatus 100 augments source training data with virtual data related to a virtual probe condition (S110). The source training data may be obtained through a simulation phantom. The imaging apparatus 100 may generate a virtual dataset with various new virtual probe conditions, adjusting parameters such as the number of sensors, the pitch between sensors, the sensor width and sensor frequency based on a source dataset.

The imaging apparatus 100 trains the neural network 200 to extract quantitative features generalized to a probe domain from input RF data using data-augmented training data, and reconstruct the generalized quantitative features to generate a quantitative ultrasound image (S120).

The neural network 200 may include an encoder 210 that extracts the quantitative feature generalized to the probe domain from the RF data, and a decoder 230 that reconstruct the quantitative feature to generate a quantitative ultrasound image. The encoder 210 may include an adaptation module 216 that generates a deformation field for deformation of a probe condition using the input RF data and a B-mode image and generalizes the quantitative feature included in the RF data using the deformation field. The adaptation module 216 may be referred to as a deformable sensor adaptation (DSA) module.

The imaging apparatus 100 may train a calibration function that generates the deformation field corresponding to the probe condition through meta-learning using the data-augmented training data. The imaging apparatus 100 may train the calibration function to minimize the difference between features deformed by the calibration function. The imaging apparatus 100 may infer the probe condition using the B-mode image together with the input RF data by the neural network 200 and train the calibration function using the inferred the probe condition. The imaging apparatus 100 may optimize and train the neural network 200 to minimize the loss of the inferred quantitative ultrasound image while performing the meta-learning of the calibration function.

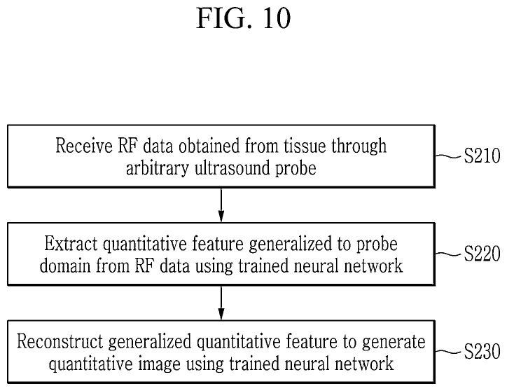

FIG. 10 is a flowchart of a probe-adaptive quantitative ultrasound imaging method according to an exemplary embodiment.

Referring to FIG. 10, the imaging apparatus 100 receives RF data obtained from tissue through an arbitrary ultrasound probe (S210). The RF data is pulse-echo data of an ultrasound signal emitted into the tissue with different beam patterns from the arbitrary ultrasound probe. The arbitrary ultrasound probe may have a sensor geometry that differs from the one used to train the neural network 200.

The imaging apparatus 100 extracts the quantitative feature generalized to the probe domain from the RF data using the trained neural network 200 (S220). The neural network 200 may include a calibration function that generates a deformation field to generalize the arbitrary probe based on the RF data and the B-mode image. The imaging apparatus 100 may generate the deformation field of the RF data by using a calibration function that performs meta-learning for probe domain generalization. The imaging apparatus 100 may generate the B-mode image from the RF data and, through the calibration function, spatially transform the probe condition inferred from a relationship between the RF data and the B-mode image into a general probe condition.

The imaging apparatus 100 reconstructs the generalized quantitative feature to generate the quantitative image using the trained neural network 200 (S230).

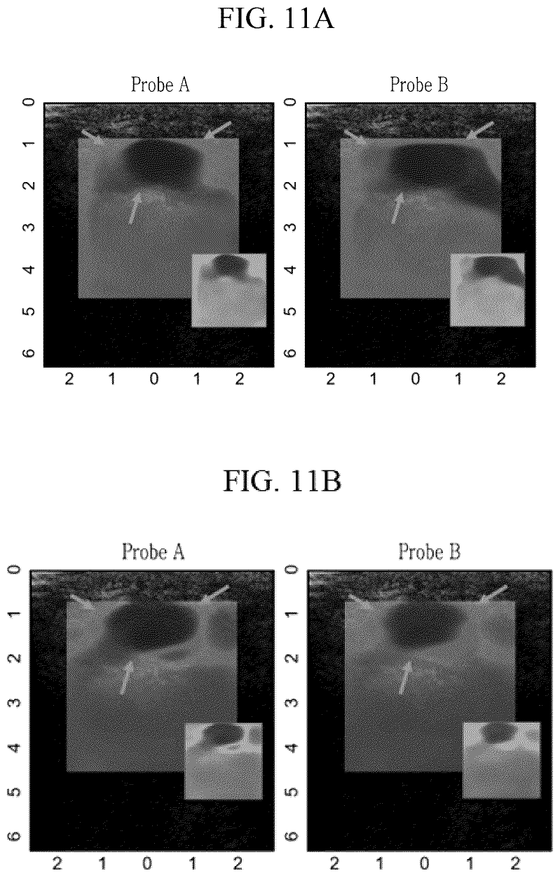

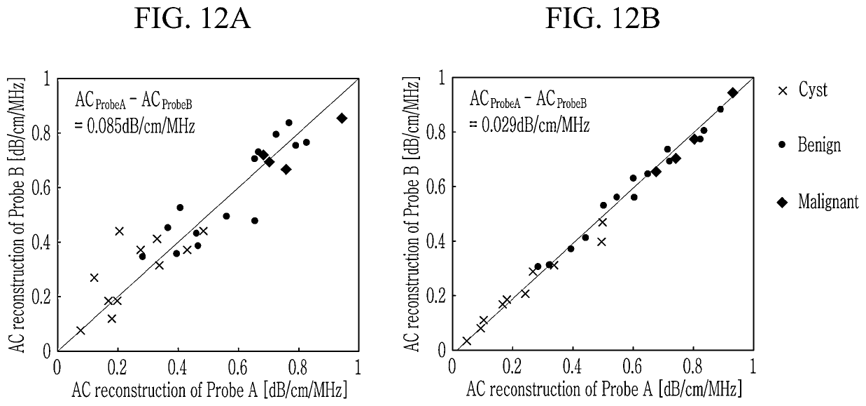

FIG. 11A and FIG. 11B illustrate quantitative imaging results, and FIG. 12A and FIG. 12B are graphs comparing the consistency of quantitative information. Referring to FIG. 11A and FIG. 11B, in vivo breast measurements are performed using probes A and B with different sensor geometries, and quantitative imaging results using the measured RF data are compared.

FIG. 11A illustrates an attenuation coefficient image generated by a comparison target model for the RF data measured by probes A and B. FIG. 11B illustrates an attenuation coefficient image generated by the imaging apparatus 100 of the present disclosure for the RF data measured by probes A and B. The comparison of the attenuation coefficient images shows that the imaging apparatus 100 may generate consistent quantitative images regardless of the probe. It shows that the imaging apparatus 100 is well generalized to unseen probes. In particular, it shows that the imaging apparatus 100 may more accurately identify the shape and attenuation coefficient value of a lesion.

Referring to FIG. 12A and FIG. 12B, a difference reconstructed attenuation coefficients of breast lesions measured by probe A and probe B is compared.

FIG. 12A illustrates an attenuation coefficient (AC) difference between breast lesions reconstructed by the comparison target model, from RF data measured by probe A and probe B. FIG. 12B illustrates an AC difference between breast lesions reconstructed by the imaging apparatus 100 of the present disclosure, from RF data measured by probe A and probe B.

It shows that the comparison target model reconstructs the attenuation coefficient depending on the probe condition. In contrast, it shows that the imaging apparatus 100 of the present disclosure can reconstruct consistent quantitative information regardless of the probe. Therefore, the imaging apparatus 100 may identify breast cancer regardless of probes, even when using the probes from various manufacturers.

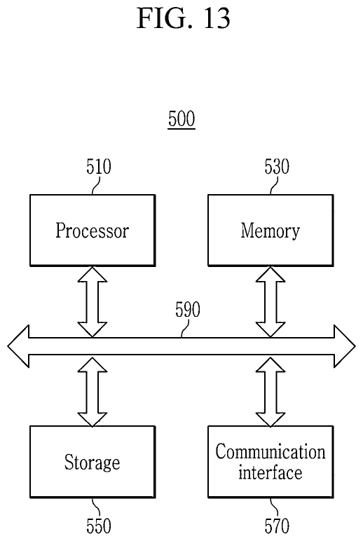

FIG. 13 is a configuration diagram of a computing device according to an exemplary embodiment.

Referring to FIG. 13, the imaging apparatus 100 may be a computing device 500 operated by at least one processor and may be connected to the ultrasound probe 10 or a device that provides data acquired from the ultrasound probe 10.

The computing device 500 may include one or more processors 510, a memory 530 that loads a program executed by the processor 510, a storage 550 that stores programs and various data, a communication interface 570, and a bus 590 connecting them. Besides, the computing device 500 may further include various components. When loaded to the memory 530, the program may include instructions that cause the processor 510 to perform methods/operations according to various exemplary embodiments of the present disclosure. That is, the processor 510 may perform the methods/operations according to various exemplary embodiments of the present disclosure by executing the instructions. The instructions are a series of computer-readable instructions grouped based on a function and indicate components of the computer program or those that are executed by the processor.

The processor 510 controls the overall operation of each component of the computing device 500. The processor 510 may be configured to include at least one of a central processing unit (CPU), a micro processor unit (MPU), a micro controller unit (MCU), a graphic processing unit (GPU), or any type of processor well-known in a technical field of the present disclosure. Further, the processor 510 may perform an operation of at least application or program for executing the method/operation according to various exemplary embodiments of the present disclosure.

The memory 530 stores various types of data, instructions, and/or information. The memory 530 may load one or more programs from the storage 550 in order to execute the method/operation according to various exemplary embodiments of the present disclosure. The memory 530 will be able to be implemented as a volatile memory such as RAM, but a technical scope of the present disclosure is not limited thereto.

The storage 550 may non-temporarily store the program. The storage 550 may be configured to include a nonvolatile memory such as a read only memory (ROM), an erasable programmable ROM (EPROM), an electrically erasable programmable ROM (EEPROM), a flash memory or the like, a hard disk, a removable disk, or any type of computer-readable recording medium well-known in the technical field to which the present disclosure pertains.

The communication interface 570 supports wired/wireless communication of the computing device 500. To this end, the communication interface 570 may be configured to include a communication module well-known in the technical field of the present disclosure.

The bus 590 provides a communication function between components of the computing device 500. The bus 590 may be implemented as various types of buses such as an address bus, a data bus, and a control bus.

As described above, according to exemplary embodiments, consistent quantitative ultrasound images can be generated regardless of probe conditions even when various types of probes are used in real-world application environments. Therefore, according to exemplary embodiments, in the field of ultrasound-based diagnostic technology, the clinical usability of quantitative information extracted based on artificial intelligence can be increased.

According to exemplary embodiments, a neural network that generates quantitative ultrasound images can be domain generalized so that the neural network can be used for a probe condition that is not seen during the training process.

According to exemplary embodiments, the quantitative ultrasound images can be generated by using various types of ultrasound probes and imaging devices for B-mode imaging as they are.

The exemplary embodiments of the present disclosure described above are not implemented only through the apparatus and the method and can be implemented through a program which realizes a function corresponding to a configuration of the exemplary embodiments of the present disclosure or a recording medium having the program recorded therein.

While the exemplary embodiments of the present disclosure have been described in connection with what is presently considered to be practical exemplary embodiments, it is to be understood that the present disclosure is not limited to the disclosed exemplary embodiments, but, on the contrary, is intended to cover various modifications and equivalent arrangements included within the spirit and scope of the appended claims.

What is claimed is:

1. An operating method of an apparatus operated by at least one processor, comprising:
receiving RF data obtained from tissue through an arbitrary ultrasound probe; and
generating a quantitative ultrasound image from the RF data using a neural network trained to perform a probe domain generalization.

2. The operating method of claim 1, wherein the generating the quantitative ultrasound image comprises:
extracting a generalized quantitative feature from the RF data using a calibration function that meta-learns probe domain generalization; and
reconstructing the generalized quantitative feature to generate the quantitative ultrasound image.

3. The operating method of claim 2, wherein the calibration function generates a deformation field spatially transforming a probe condition of the arbitrary ultrasound probe to a generalized probe condition.

4. The operating method of claim 3, wherein the generating the quantitative ultrasound image comprises
applying the deformation field generated by the calibration function to a feature of the RF data to generate a deformed feature to the generalized probe condition.

5. The operating method of claim 2, further comprising
generating a B-mode image from the RF data,
wherein the generating the quantitative ultrasound image comprises
generalizing the probe condition inferred from a relationship between the RF data and the B-mode image, using the calibration function; and
extracting the generalized quantitative feature from the RF data.

6. The operating method of claim 1, wherein the quantitative ultrasound image includes quantitative information for at least one parameter among speed of sound (SoS), attenuation coefficient (AC), effective scatterer Concentration (ESC), and effective scatterer diameter (ESD).

7. The operating method of claim 1, wherein the neural network is an artificial intelligence model trained to generalize the probe domain of input RF data using training data augmented with virtual probe conditions.

8. An operating method of an apparatus operated by at least one processor, comprising:
augmenting source training data with virtual data related to virtual probe conditions; and
training a neural network using data-augmented training data, the neural network trained to generate a quantitative ultrasound image from an input RF data by a probe domain generalization.

9. The operating method of claim 8, wherein the augmenting the source training data comprises
generating new virtual data by changing at least one of the number of sensors of a probe, a pitch between sensors, a sensor width and sensor frequency in the source training data.

10. The operating method of claim 8, wherein the training the neural network comprises
training a calibration function in the neural network, through meta-learning using the data-augmented training data, the calibration function configured to generate a deformation field corresponding to a probe condition of the input RF data; and
wherein the calibration function is trained to generates the deformation field spatially transforming a probe condition of an arbitrary ultrasound probe to a generalized probe condition.

11. The operating method of claim 10, wherein the calibration function performs the meta-learning that generates the deformation fields spatially transforming the probe condition of the input RF data to a generalized probe condition, based on a relationship between the input RF data and a B-mode image generated from the input RF data.

12. The operating method of claim 10, wherein the training the neural network comprises
training the neutral network to minimize a loss of an interfered quantitative ultrasound image while performing the meta-learning of the calibration function.

13. The operating method of claim 8, wherein the neural network includes:
an encoder extracting a quantitative feature generalized to a probe domain from the input RF data using an adaptation module that generalizes the probe condition of the input RF data; and
a decoder reconstructing the generalized quantitative feature to generate the quantitative ultrasound image.

14. An imaging apparatus comprising:
a memory; and
a processor executing instructions loaded to the memory,
wherein the processor is configured to:
receive RF data obtained from tissue through an arbitrary ultrasound probe, and
generate a quantitative ultrasound image by performing a probe domain generalization for the RF data using a trained neural network.

15. The imaging apparatus of claim 14, wherein the processor is configured to:
extract a generalized quantitative feature from the RF data using a calibration function that meta-learns probe domain generalization; and
reconstruct the generalized quantitative feature to generate the quantitative ultrasound image.

16. The imaging apparatus of claim 15, wherein the calibration function generates a deformation field spatially transforming a probe condition of the arbitrary ultrasound probe to a generalized probe condition.

17. The imaging apparatus of claim 15, wherein the processor is configured to apply the deformation field generated by the calibration function to a feature of the RF data to generate a deformed feature to the generalized probe condition.

18. The imaging apparatus of claim 15, wherein the processor is configured to:
generate a B-mode image from the RF data; and
generalize a probe condition inferred from a relationship between the RF data and the B-mode image using the calibration function, and then extract the generalized quantitative feature from the RF data.

19. The imaging apparatus of claim 14, wherein the quantitative ultrasound image includes quantitative information for at least one parameter among speed of sound (SoS), attenuation coefficient (AC), effective scatterer Concentration (ESC), and effective scatterer diameter (ESD).

20. The imaging apparatus of claim 14, wherein the neural network includes:
an encoder extracting a quantitative feature generalized to a probe domain from the input RF data using an adaptation module that generalizes the probe condition of the input RF data; and
a decoder reconstructing the generalized quantitative feature to generate the quantitative ultrasound image.
